(19) United States
(12) Patent Application Publication
(10) Pub. No.: US 2010/0022990 A1
(43) Pub. Date: Jan. 28, 2010

(54) PUMP SYSTEM FOR NEGATIVE PRESSURE WOUND THERAPY AND IMPROVEMENTS THEREON

(75) Inventors: John Karpowicz, Chester Springs, PA (US); Kevin P. Klocek, Wynnewood, PA (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: BOEHRINGER TECHNOLOGIES, L.P., Norristown, PA (US)

(21) Appl. No.: 12/507,846

(22) Filed: Jul. 23, 2009

Related U.S. Application Data
(60) Provisional application No. 61/083,676, filed on Jul. 25, 2008.

(51) Int. Cl. A61M 27/00

(52) U.S. Cl. 604/543

(57) ABSTRACT

A wound therapy system is provided which includes at least one sensor placed in a wound for sensing information regarding status of a body of a living being, and a communication link for electronically passing the information regarding the at least one sensor to a controller. A method for providing wound therapy is also provided which includes the steps of providing at least one sensor, each sensor placed in a wound, sensing information regarding status of a body of a living being utilizing the at least one sensor, and passing the information from the at least one sensor to a controller, via a communication link, between the at least one sensor and the controller.

This application claims priority to U.S. Provisional Patent Application No. 61/083,676, entitled PUMP SYSTEM FOR NEGATIVE PRESSURE WOUND THERAPY AND IMPROVEMENTS THEREON, filed on Jul. 25, 2008.

### BACKGROUND OF THE INVENTION

The disclosed invention relates to improvements to devices and methods for treating wounds with negative pressure and, specifically, the controls, monitors and alarms that optimize the process of wound healing.

Suction in a hospital setting is an important adjunct to therapy of many types. Wound drainage and removal of exudates is a common use of suction. In addition to the removal of fluids, suction is believed to enhance the healing characteristics of many wounds. Negative pressure wound therapy is an important modality to assist in the healing of chronic and acute wounds.

Current technology is defective in that critical wound variables cannot be measured with current patient treatment equipment. While many variables could be measured in the fluid that is collected into waste receptacles, certain variables are best measured in the wound directly, such as temperature and pH.

Optimum delivery of negative pressure wound therapy (NPWT) depends on the consistent application of negative pressure. Prior applications and patents by one or more of the present inventors have addressed the importance of pressure monitoring and have described flow based detection methods and devices. These include, for example, U.S. Patent Application Publication No. 2008/0132819 (Radl et al.) which shows a tunnel dressing for use with a NPWT system, U.S. Patent Application Publication No. 2006/0025727 (Boehringer et al.) which teaches an apparatus and method for suction-assisted wound healing, U.S. Patent Application Publication No. 2005/0209574 (Boehringer et al.) which teaches a wound packing material for use with suction, U.S. Pat. No. 7,485,112 (Karpowicz et al.) and U.S. Patent Application Publication No. 2009/0131892 (Karpowicz et al.) which teach a tube attachment device for wound treatment, U.S. Patent Application No. 2009/0137973 (Karpowicz et al.) which teaches a system for treating a wound with suction and a method of detecting loss of suction, U.S. Patent Application Publication No. 2009/0012501 (Boehringer et al.) which teaches a system for suction-assisted wound healing, U.S. Patent Application Publication No. 2009/0005744 (Karpowicz et al.) which teaches a system for treating a wound with suction and a method of detecting loss of suction, U.S. Pat. No. 7,438,705 (Karpowicz et al.) which teaches a system for treating a wound with suction and a method of detecting loss of suction, U.S. Patent Application Publication No. 2007/0219532 (Karpowicz et al.) which teaches a pump system for negative pressure wound therapy, U.S. Patent Application Publication No. 2008/0177253 (Boehringer et al.) which teaches a growth stimulating wound dressing with improved contact surfaces, and U.S. Patent Application Publication No. 2008/0005000 (Radl et al.) which teaches a billing method for a NPWT system.

While control of pressure and flow are essential to the monitoring of the NPWT process, they do not provide direct indication of the underlying healing process. Improvements in sensor technology enable direct measurement of negative pressure within the wound as well as provide the ability to characterize the wound environment for the presence of beneficial and/or detrimental conditions. Monitoring of the microenvironment of the wound is useful in assessing the overall healing process and further optimizing the role of negative pressure wound therapy. Remote reporting of these performance attributes to the clinical staff will improve quality of care.

All references cited herein are incorporated herein by reference in their entireties.

### BRIEF SUMMARY OF THE INVENTION

The invention is directed to a wound therapy system having a pump that supplies controlled negative pressure to a dressing that is applied to a wound of a patient. The wound therapy system preferably includes an array of sensors for monitoring the condition of the wound. The array of sensors may include sensors for monitoring pressure or suction throughout the wound, psychometric sensors for monitoring temperature and relative humidity at the surface of the wound, sensors for monitoring pH, oxygen and CO₂ at the surface of the wound, sensors for quantifying the presence of beneficial metabolites as would be an indicator of normal wound healing at the wound surface or in the effluent that emanates from the wound, sensors for quantifying the presence of detrimental metabolites as would be an indicator of impaired wound healing or as an indicator of urgent action required at the wound surface or in the effluent emanating from the wound, and/or sensors for monitoring blood flow in the tissue at the surface of the wound.

The system may be adaptable to add metabolites that are to be detected and the sensors may communicate with a host device for data acquisition and closed loop control. The host device may communicate with delivery actuators to deliver agents that can correct abnormal conditions detected in the wound microenvironment. The host device may communicate with an external data server to relay patient data and patient compliance for monitoring of system performance. A pump for adding metabolites could be a peristaltic pump or syringe pump, or drip infusion could be used to instill metabolites down a second lumen directed to the wound. Similarly, gravity, in combination with the negative pressure at the wound bed could draw materials into the wound bed. A solenoid could be used to set the timed rate of infusion

Other desirable features of the present invention may include providing real time monitoring of in-vivo conditions of the wound environment, providing current information on the actual level of suction or pressure at the surface of the wound, providing current information on other physiologic parameters such as temperature of the wound and surrounding tissue as well as the relative humidity in the wound cavity, providing information on the physiochemical aspects of the wound environment such as pH, oxygen and CO₂ levels for assessing the stage of healing, measuring trace levels of metabolic byproducts that indicate a healthy healing environment such as that given off by tissue macrophages and fibroblasts, measuring trace levels of metabolic byproducts that indicate an unhealthy healing environment such as infectious byproducts, measuring the development of blood flow to the tissues in the bed of the wound and adjacent the wound to determine that therapy is beneficial, providing the above information using a micro sensor array that is embedded into the wound dressing and where the information is transmitted wirelessly or via a flexible cable attached to a monitoring system, providing the above information using a micro sensor array that is placed into the wound cavity and where the information is transmitted wirelessly or via a flexible cable attached to a monitoring system. The system may preferably manage and integrate the above information into revised treatment protocols. The system may also relay information to a central remote station for performance monitoring.

In a preferred embodiment of the present invention, a wound therapy system is provided that includes at least one sensor, placed in a wound, for sensing information regarding status of a body of a living being and a communication link for electronically passing the information regarding the at least one sensor to a controller.

Preferably, the at least one sensor is a pressure sensor to sense pressure, a suction sensor for sensing suction, a temperature sensor to sense temperature, a relative humidity sensor to sense relative humidity, a pH sensor to sense a level of pH, an oxygen sensor to sense a level of oxygen, a CO₂ sensor to sense a level of CO₂, and/or a blood flow sensor for monitoring blood flow in tissue at the surface of the wound. The controller may provide real time monitoring of conditions sensed by the at least one sensor in the wound and may control a level of negative pressure applied to the wound. A sensor may be provided for quantifying the presence of beneficial metabolites as an indicator of normal wound healing. A pump and a conduit from the pump to the wound may be provided for adding metabolites. The information from the sensors may be used in a feedback system in the controller to control the pump for adding metabolites in the controller based on information from the at least one sensor. A solenoid may be provided for setting a timed rate of infusion. One of the sensors included may be a collagen sensor for quantifying the rate of tissue regeneration in the wound. One of the sensors may be a sensor for quantifying the presence of detrimental metabolites as an indicator of impaired wound healing or as an indicator of urgent action required. One or more of the sensors may be an organic compound sensor and/or an inorganic compound sensor.

The communication link may be a wireless transmitter or may use wires. The sensor may be an array of sensors. The controller may be capable of receiving and interpreting signals from a global positioning system to allow it to determine an instantaneous location of the controller.

The controller may also include a transmitter to communicate with a central server to relay patient information. The transmitter may be wired or wireless. The patient information transmitted to the central server may be information such as instantaneous patient compliance, historical patient compliance, error messages, and service related issues. The controller may include a receiver to receive downloaded information from the central server, wherein the information is information such as updated operating software, clinical operating protocols, and user defined settings. Finally, the transmitter may communicate with the central server to relay compliance time, wherein the compliance time is a period of time wherein clinically effective levels of suction are delivered to the patient's wound bed.

In another embodiment of the invention, a wound therapy system is provided that includes at least one sensor for sensing information regarding status of a body of a living being, a controller for monitoring and interpreting the information wherein the information is a pressure in the wound, a communication link for electronically passing the pressure in the wound to the controller, and wherein the controller includes a transmitter to communicate the information to a central server. The pressure transmitted may be a pressure that has been maintained in the wound over a predetermined period of time.

A method for providing wound therapy is also provided which includes the steps of providing at least one sensor, each sensor placed in a wound, sensing information regarding status of a body of a living being utilizing the at least one sensor, and passing the information from the at least one sensor to a controller via a communication link between the at least one sensor and the controller.

The step of providing at least one sensor may include providing a pressure sensor to sense pressure, a suction sensor for sensing suction, a temperature sensor to sense temperature, a relative humidity sensor to sense relative humidity, a pH sensor to sense a level of pH, an oxygen sensor to sense a level of oxygen, a CO₂ sensor to sense a level of CO₂, and a blood flow sensor for monitoring blood flow in tissue at the surface of the wound. A step of real time monitoring of conditions sensed by the at least one sensor in the wound may be provided.

The step of providing at least one sensor may include providing a sensor for quantifying the presence of beneficial metabolites as an indicator of normal wound healing. The step of adding metabolites may include pumping the metabolites through a conduit to the wound. A step of providing a feedback system in the controller wherein the information is used in a feedback system in the controller to control adding the metabolites in the controller based on information from the at least one sensor may be provided. A step of setting a timed rate of infusion of by operating the pump may be included.

The step of providing at least one sensor may include providing a collagen sensor for quantifying the rate of tissue regeneration in the wound. The step of providing one of the at least one sensor may include providing a sensor for quantifying the presence of detrimental metabolites as an indicator of impaired wound healing or as an indicator of urgent action required.

The step of providing one of the at least one sensor may include providing an organic compound sensor. The step of providing one of the at least one sensor may include providing an inorganic compound sensor.

The step of passing the information from the at least one sensor to a controller may include wirelessly transmitting the information from the at least one sensor to the controller or passing the information via wires.

The step of providing at least one sensor may include providing an array of sensors.

The method may further include step of providing the controller which is capable of receiving and interpreting signals from a global positioning system and the step of determining an instantaneous location of the controller.

The method may also include the step of transmitting patient information to a central server, such as instantaneous patient compliance, historical patient compliance, error messages, and controller or sensor service. The method may also include the step of downloading information from a central server to the controller, such as updated operating software, clinical operating protocols, and user defined settings. The method may also include the step of transmitting compliance time, wherein the compliance time is a period of time wherein clinically effective levels of suction are delivered to the patient's wound bed. The step of transmitting compliance time may include transmitting a value equal to compliance time divided by a selected time period where the wound therapy system is operating.

In another embodiment of the present invention, a method for providing wound therapy is provided that includes the steps of providing at least one sensor, sensing information regarding status of a body of a living being utilizing the at least one sensor, passing the information from the at least one sensor to a controller via a communication link between the at least one sensor and the controller, wherein the controller is for monitoring and interpreting said information and wherein said information is the pressure in the wound, and transmitting patient information from the controller to a central server. The information may be a pressure that has been maintained in the wound over a predetermined period of time.

### BRIEF DESCRIPTION OF SEVERAL VIEWS OF THE DRAWINGS

The invention will be described in conjunction with the following drawings in which like reference numerals designate like elements and wherein:

FIG. 1 is a simplified exploded isometric view of a wound therapy system having a wireless system having components for utilizing sensors in a body cavity to transmit information on the status of the environment in the body cavity, in accordance with a preferred embodiment of the present invention;

FIG. 2 is a simplified isometric view of the components of a wire-based wound therapy system in accordance with another preferred embodiment of the present invention;

FIG. 3 is a block diagram of a wound therapy controller (wired or wireless) for the wound therapy systems of FIGS. 1 and 2; and

FIG. 4 is a simplified schematic view of a wound therapy system showing a layout of the transmitter components of FIGS. 1-3.

### DETAILED DESCRIPTION OF THE INVENTION

The wound environment as it pertains to healing is complex and, therefore, lends itself to monitoring that is indicative of environmental conditions that are known to lead to favorable outcomes. For moist wound healing involving negative pressure therapy, it is desirable to know the levels of pressure, temperature, relative humidity and pH at the wound. Maintaining these environmental conditions is well known to have a beneficial impact on the healing process. There is heretofore no reliable method to assess these environmental conditions in the actual wound environment, much less in the environment of NPWT.

There are also indicators of a wound that are more indicative of a favorable or unfavorable metabolic process. A coarse indicator of such an indication is odor. A wound that is infected will have a noticeable odor indicative of decaying flesh and bacterial proliferation. The presence of trace amounts of metabolic byproducts indicative of a favorable or unfavorable metabolic process would be useful information to the caregiver regarding the course of action on a particular wound. For example, a trace amount of hydrogen sulfide would indicate that there is a potential for infection and would serve as an alert to the caregiver to proactively treat the infection either topically or systemically or both. Such a system could have great potential advantage in anticipating potential situations that would delay the healing process. An infective process may well be underway in the wound microenvironment and the coarse indicator of odor occurs well after significant colonization takes place. In treatment of chronic wounds, such as a decubitus ulcer overlying the sacrum, fecal contamination occurs and can significantly impact the healing process. Other wounds in the abdominal cavity may become infiltrated with other body fluids that are detrimental to the healing process such as stomach bile or intestinal fluids as may be encountered if there are active exuding fistulae.

Micro-sensor arrays are becoming a practical solution for monitoring numerous environments as well as a wide array of conditions. The Georgia Institute of Technology has developed an array for measuring volatile organics. See Georgia Institute of Technology, New Microsensor Measures Volatile Organic Compounds in Water and Air (Sep. 18, 2007). Mahaveer et al. describe wireless transmission of pH, temperature and pressure using a micro sensor array. See Mahaveer K Jain et al., “A Wireless Micro-Sensor for Simultaneous Measurement of pH, Temperature, and Pressure,” 2001 Smart Mater. Struct. 10 at pages 347-353, doi:10.1088/0964-1726/10/2/322. See also “Micro-Sensor Array Platform Fact Sheet,” by the Oak Ridge National Laboratory at www.Ornl.gov.

The present invention is directed to an array of sensors placed directly into the wound that wirelessly (or, less preferably, using wires) relay information on the environment of the wound and the presence of key biochemical markers that are crucial to effective healing. Wireless technology enhances the utility of such devices.

ISFET's are micro circuits with applicability for monitoring pH. Locating an array of these circuits at the wound contact interface would allow the host device to determine the pH at the wound as a predictor of positive or negative progress of wound healing. A collagen sensor could be used to quantify the rate of tissue regeneration in the wound bed and the transition to the proliferative phase of healing. Sensors such as this type are described by Swatland et al. in “UV Fiber Optic Probe Measurements of Connective Tissue in Beef Correlated with Taste Panel Scores of Chewiness,” H. J. Swatland, E Gullett, T. Hore and S. Buttenham, Food Research International, Vol. 28, Issue 1, 23-30. Laser Doppler or LED emitter/receiver arrays could be placed at the wound contact interface to allow for monitoring of topical perfusion and tissue oxygenation. This information would allow for adjustments to overall therapy based on adequate therapy being delivered to the wound bed.

The invention will be illustrated in more detail with reference to the following embodiments, but it should be understood that the present invention is not deemed to be limited thereto.

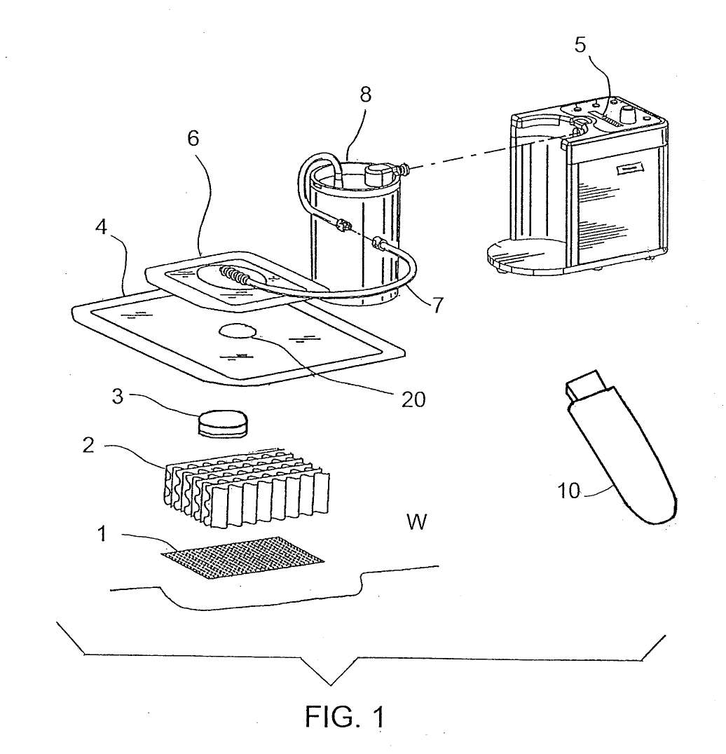

Referring to FIG. 1, shown is wound W, which is to be treated with negative pressure therapy. The wound surface is covered with a contact layer 1 and the wound cavity is filled with packing 2. Contact layer 1 is described, for example, in U.S. Patent Application Publication Nos. 2005/0209574 Boehringer et al.) and 2008/0177253 (Boehringer et al.). Packing 2 is described, for example, in U.S. Patent Application Publication No. 2005/0228329 (Boehringer et al.). The general practice is to cut the contact layer 1 and the packing 2 to just fit within the wound space, ensuring that the materials contact efficiently all wound surfaces and that the wound space is generally filled without over packing the wound W.

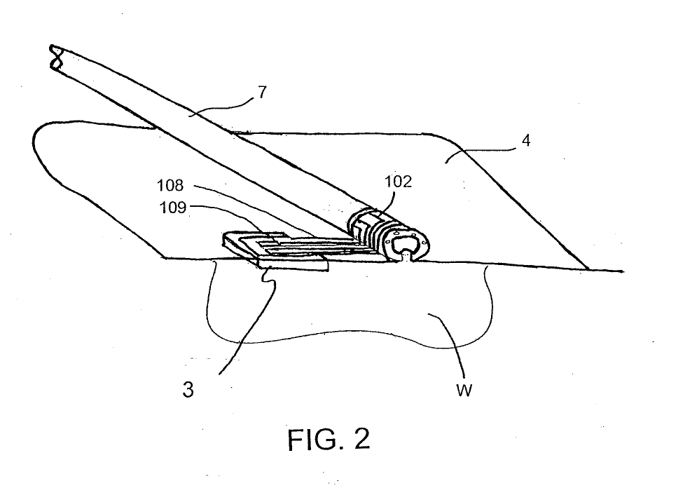

It is at this point that a wound sensor array 3 is placed into the wound space. The wound sensor array 3 is a disc shaped device that has internal power source, select sensor technologies and is wired or has a wireless transmitter for communication capability for conveying key information back to the wound therapy controller 5. This information is preferably transmitted wirelessly (as shown in FIG. 1), but may also be wired, as desired or as circumstances require, as will be discussed with respect to FIG. 2. Prior to placement in the wound W, the wound sensor array 3 is linked to the wound therapy controller 5 through a coding sequence to ensure that the wound therapy controller 5 is correctly interpreting information from the proper sensor array 3.

The wound W is then covered with cover 4 to seal the wound from the outside environment. Wound cover 4 is typically a thin film material that has sufficient water vapor permeability to allow moisture to migrate away from the wound for comfort and healing benefits. Thicker materials may be employed insofar as moisture is permitted to migrate away from the wound to avoid skin maceration and wound degradation. A hole 20 is placed in the cover 4 to allow wound fluids to be conveyed out of the wound W.

A coupling 6 is placed over the hole 20 in cover 4 and provides the means for delivering negative pressure to the wound and conveying materials away from the wound W. The coupling 6 may be seen, for example, in U.S. Pat. No. 7,485,112 (Karpowicz et al.) and U.S. Patent Publication No. 2009/0131892 (Karpowicz et al.).

The coupling 6 includes conduit 7 which communicates with collection vessel 8 for the accumulation of fluids from the wound.

Canister 8 preferably inserts into a receiver in the wound therapy controller 5 where a source of negative pressure is supplied from an internal pump mechanism.

The prior art includes control algorithms that are used to maintain a controlled level of negative pressure determined by user applied set points. See, for example, U.S. Pat. No. 7,438,705 (Karpowicz et al.) and U.S. Patent Application Nos. 2007/0219532 (Karpowicz et al.) and 2009/1037973 (Karpowicz et al.). This approach suffers from the limitation that pressures must be sensed remotely and are subject to clogging and occlusions that affect the true and correct pressure reading.

In the present invention, the wound therapy controller 5 has a communication capability that links with the transmitter of the wound sensor array 3 that is placed in the wound W. While this communication link could be achieved by hard wire transmission, radio transmission is preferable (but not required in accordance with the present invention). Any known type of communication is intended to be within the scope of the present invention. The user selects a negative pressure therapy setting to be maintained and the wound therapy controller 5 administers negative pressure to the system until the predetermined pressure is reached in the wound W. A control algorithm maintains this pressure within prescribed limits. The wound therapy controller 5 periodically receives data transmission from the transmitter and responds by turning on the pump of the wound therapy controller 5, if appropriate.

In this fashion, negative pressure may be effectively maintained in the wound environment while greatly minimizing the inherent limitations in direct pressure measurement and flow or leak measurement systems. While pressure maintenance is an example of a feedback closed loop control system, other variables such as pH and temperature can be measured and this information can used in a control algorithm to supply a corrective action.

Referring to FIG. 2, a wire based system in accordance with the present invention is illustrated that provides the same type of monitoring capability as the wireless based system previously described with respect to FIG. 1. The components of FIG. 2 will be described as they apply to FIG. 1, but the system is wired, rather than wireless. To overcome the size and cost potentially associated with batteries, insulated wires or conductive paths are preferably integrated into conduit 7 (see FIG. 1 also). A sensor array 3 is shown on top of wound W. Conductors 102 transverse the length of conduit 7 and are oriented on the top quadrants of the conduit 7. The conduit 7 surface is removed to expose the conductors 102 and a ribbon cable 108 is affixed to the conduit 7 and conductors 102, establishing an array of conductive paths that transverse cover 4 (see also FIG. 1). The sensor array 3 attaches through cover 4 to the terminal ends of the ribbon cable 108. While only four conductive paths 109 are illustrated, this number is not intended to be limited and can include as many as are capable of existing based on miniature wiring techniques. Thus, in this fashion, it is feasible to supply power and obtain signals from any number of transducers that are placed in the wound cavity W. Other embodiments include a Siamese tube, wherein the conductive wires are placed in one of the lumens (not shown).

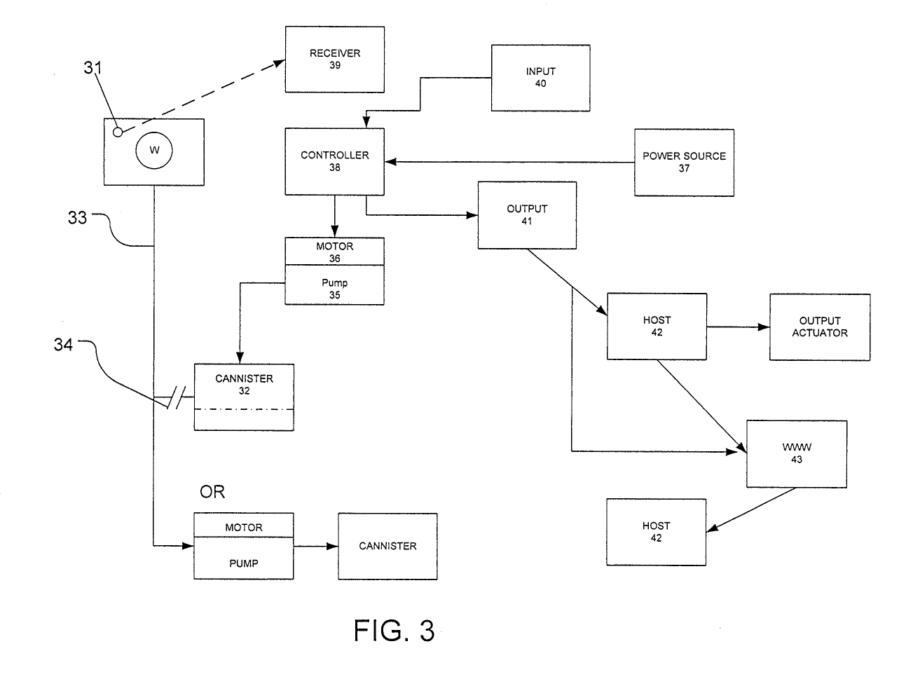

Referring to the block diagram of FIG. 3, wound cavity W is covered and sealed as previously described to maintain an essentially airtight cavity, within which data transmitter 31 has been placed. Wound cavity W is fluidly connected to a collection canister 32 using tubing 33 as such is produced commonly from polyvinylchloride. Connectors 34 are optionally employed to disconnect the system from the wound as may from time to time be advantageous. Canister 32 is of typical waste canister construction with features to ensure that fluids are effectively contained as in an overflow condition. Hydrophobic membranes are routinely used for this purpose. Canister construction is preferably performed to provide a hermetic, leak free construction. Canister 32 is fluidly connected to a pump 35. Pump 35 is preferably a positive displacement type of pump employing a diaphragm and inlet and outlet valves to reliably supply negative pressure to the collection circuit. Pump 35 is driven by a motor 36, preferably a low voltage DC type that is readily powered by a power source 37 such as a battery pack. Rechargeable batteries are readily adapted to the device. Control Module 38 (also called controller 38) is typically a microprocessor device that accepts data information from receiver 39 that is in wireless communication with the transmitter that has been embedded in wound cavity 30. Receiver 39 may also be in direct communication via wires as previously described. Receiver 39 collects relevant data from transmitter such as the local pressure, temperature, humidity levels etc. within the wound cavity 30 and control module 38 provides input to pump 35 and motor 36 to turn on in order to maintain the predetermined local negative pressure. User interface 40 (also called input 40) may include a keypad for selecting device functions and environmental conditions. Other input mechanisms are anticipated such as wireless type devices or a web based communication system. Output 41 preferably consists of a USB compatible device for exporting data to a memory card or for direct attachment to a host device 42 such as a computer either directly or via the internet 43.

The devices and methods described herein are readily adapted to provide telemetric monitoring of a body cavity, absent the application of negative pressure wound therapy. The wireless sensor array may be configured to be placed within a wound that is not under negative pressure, and the sensor can still function to relay important information on the wound microenvironment. There are other body cavities, such as the abdomen, where remote sensing may be even more useful for deep cavity wounds where vacuum is employed.

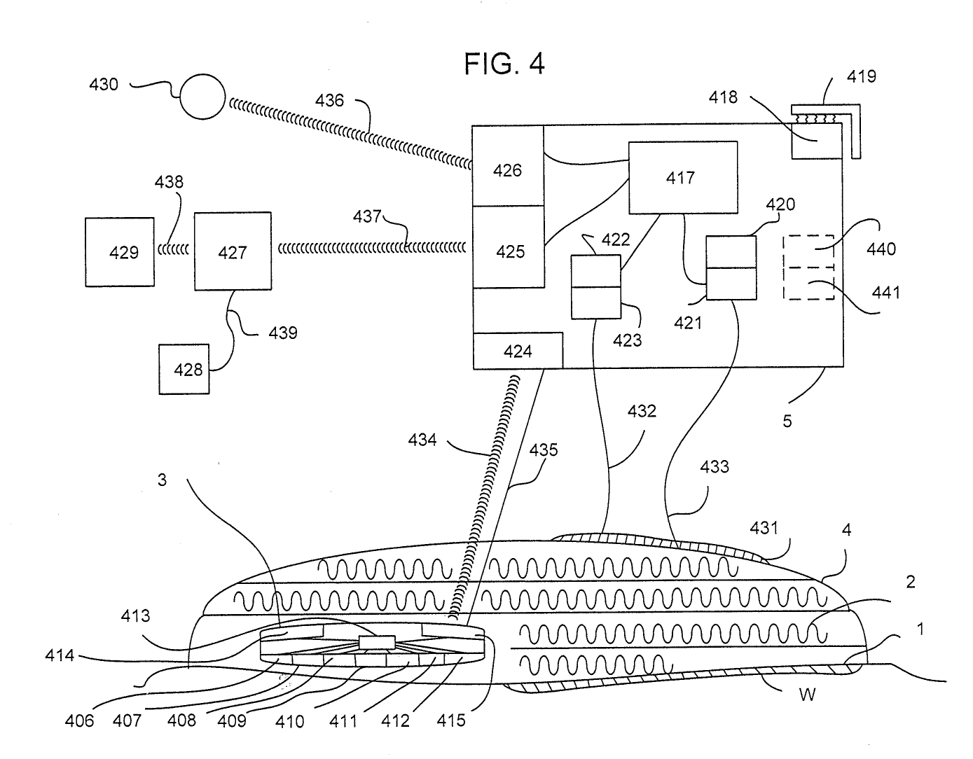

Referring now to FIG. 4, which shows a simplified schematic view of a wound therapy system showing a layout of the transmitter components in accordance with the present invention, patient wound bed W is covered with an appropriate wound contact layer 1. An appropriate amount of wound packing material 2 is used to fill in the wound deficit. A wound cover 4 creates an area underneath which in appropriate amount of negative pressure may be maintained. A sensor array 3 is placed underneath wound cover 4 and may be in intimate contact with patient wound bed W. This sensor array 3 is comprised of a number of distinct elements which may be used in whole or in part. A sensor microcontroller 413 facilitates the input and output of information from the sensor array 3. A sensor battery 414 powers the sensor array 3. The sensor array 3 has the following discrete sensors embedded in the device: a pressure sensor 406, a pH sensor 407, a temperature sensor 408, a humidity sensor 409, a perfusion sensor 410, a tissue oxygenation sensor 411, and a beneficial metabolite sensor 412. A wound therapy controller 5 works in communication with the sensor array 3, and is programmed to receive signals from singular or multiple sensor arrays 3 placed in the same or different patient wound beds W on the same patient. The sensor array 3 communicates with the wound therapy controller 5 either via sensor wireless transmitter 415 facilitating sensor array wireless communications 434. Optionally, communication can be facilitated with sensor array wired communications 435. The wound therapy controller 5receives communication from the sensor array 3 via the therapy unit local antenna 424.

Examples of sensors that would likely operate acceptably include a pressure sensor by Honeywell, part number 24PCC, a pressure sensor by Silicon Microstructures, Inc., part number SM5102, a humidity and temperature sensor by Sensirion, part numbers SHT10, SHT11, SHT15, a detrimental metabolite (hydrogen sulfide) sensor by Alphasense, Ltd., part number H2S-A1, a sensor capable of measuring pH such as by Microsens, part number MSFET 3310, a probe for measuring oxygen/perfusion and temperature by Discovery Technology International, LLLP, a temperature sensor by Burr Brown Products from Texas Instruments, and part number TMP141.

The wound therapy controller 5 is principally controlled via the therapy unit microcontroller 417. The wound therapy controller 5 is powered via an internal power source, therapy unit battery 418. An external noncontact RF charging coupler for therapy unit 419 may be used to provide wireless power and recharging capability for the device to allow for portability. Based on inputs from the sensor array 3 and the desired user setpoints, the therapy unit microcontroller can operate the suction pump 422. Suction created from this pump is applied to the patient wound bed W via suction delivery tube 432 which penetrates the wound cover 4 through tube attachment device 431. Gaseous sensor 423 is connected upstream or downstream of the pump to allow sampling of the gaseous components of exudate removed from patient wound bed W through suction delivery tube 432.

Medications or other beneficial wound healing agents may be instilled to the patient wound bed W though instillation tube 433. Fluid reservoir 420 contains these beneficial agents. Fluid delivery actuator 421 allows for the control of these beneficial agents to the wound bed. Fluid delivery actuator 421 can take the form of a solenoid to allow for gravity and suction instillation, or alternatively it may be a positive displacement pump. It is anticipated the multiple agents may be additionally instilled for a particular healing benefit, these would be described by secondary fluid reservoir 440 and secondary delivery actuator 441 which would function in parallel to items 420 and 441.

A global positioning system (GPS) may also be incorporated into the present invention, as will be discussed in greater detail below. GPS antenna 426 receives GPS transmissions signals 436 from the GPS source 430. These signals are interpreted by the therapy unit microcontroller 417 to determine the present physical location of the therapy unit.

The wound therapy controller 5 communicates with a server 427 via therapy unit wireless communications 437 distributed by therapy unit transmission antenna 425. These signals can be GSM, GPRS, or WiFi dependant on the network communications protocol utilized between therapy unit 417 and server 427.

Server 427 can communicate to a local paging device 429 via central server wireless communications 438. Server 427 may also relay information via central server wired communications 439 to a terminal 428. This would allow the server to relay patient information.

The wound therapy controller may be equipped with a host connection (Universal Serial Bus type A, as determined by USB-IF) or a peripheral connection (USB type B). These connections allow the unit to write to and read from a portable memory devices, or connect to a personal computer for the purpose of uploading or downloading information.

The wound therapy controller 5 may be capable of receiving and interpreting signals from the Global Positioning System (United States Department of Defense NAVSTAR system) to allow it to determine its instantaneous location. This may aid in billing particular patients and serves to prevent wound therapy systems reimbursed in one care environment from being improperly used in another care environment. GPS could also be used to locate the patient to provide continued or emergent treatment if needed.

For GPS information to be useful the wound therapy controller 5 must have a way of sending this information to a remote server, such as a transmitter. There are a variety of transmission techniques which would allow the unit to communicate with an external server.

The wound therapy controller 5 could use a wide area network such as GPRS (3rd Generation Partnership Project TS 26.233 and TS 26.234). Another suitable standard would be far field WiFi (IEEE 802.11b, 802.11g and 802.11n). Another suitable option would be near field Bluetooth (ISO/IEC 26907 and 26908) or Zigbee (IEEE 802.15.4). Many of these transmission features can be found in a modular device such as Telit Industries GE863-GPS. These features can also be replicated singularly with discrete hardware and logic. This would allow the following capabilities—

Upload from host device (i.e., the wound therapy controller 5) to external server—
Instantaneous patient compliance
Historical patient compliance
Recorded errors messages
Service information from the unit (performance issues with the internal battery, suction pump, or power handling components.)

Download from remote server to a receiver on the host device (i.e., the wound therapy controller 5)—
Updated operating software
Specifically indicated clinical operating protocols (such that therapy could be varied in a defined way over the next few days, weeks, months as prescribed by a clinician for a particular clinical benefit) pressure level, intermit time and duty cycle.
User defined settings such as alarm intensity, indicator intensity. Setting lock features.

The addition of wireless transmission could allow for status alarms to be relayed to a central station monitoring system or an offsite clinician. The ability to communicate with the unit could also allow an off-site caregiver to reinitiate therapy if it has been inadvertently interrupted by the patient.

Particular types of building construction cause interference with GPS signals. The inability of the GPS receiver to see the GPS satellites could cause an inappropriate estimation of the unit's location. One particular solution is to rely on triangulation of existing cellular phone signals, these protocols are defined by ISO/IEC 24730-1,2,5. Similarly known static WiFi locations could be triangulated by the onboard antenna of the pump. A final solution is to have the unit report its last known location across the wireless network and then scan for an acceptable GPS signal. The remote server would know the last known location of the host device before it lost its GPS signal.

Wound therapy systems of this type are typically used for periods of up to four months and typically 24 hours per day. They see varied environmental conditions and may need to be repeatedly cleaned to prevent the cross contamination of device from patient to patient. It would be preferable to have a unit which is easily cleanable by a variety of methods including wiping or spraying with an effective hospital disinfectant such as 3M Quat® or Bleach. Previous designs have shown that the fluid path can be sealed from the internal electronics of the unit, but it is also important to seal the internal electronics from outside user interfaces. The inclusion of wireless communication could allow the pump to send data and receive external inputs without the need for a user keypad or electrical contacts which could create a path for unwanted electrostatic discharge or foreign debris to accumulate.

The skin of the therapy device could utilize a printed organic transistor material that would allow the device to be recharged absent any exposed electrical connections. This would mean the outside of the device would be free of any electrical contacts or means to trap foreign debris. With the ability to send and receive patient data as well as the ability to recharge itself wirelessly, the entire unit could be hermetically sealed for ease of cleanability and to improve infection control measures. See “A Large-Area Wireless Power-Transmission Sheet Using Printed Organic Transistors and Plastic MEMS Switches. Tsuyoshi Sekitani, Makoto Takamiya, Yohsiaki Noguchi, Shintaro Nakano, Yusaku Kato, Takayasu Akayasu Sakurai and Takao Someya, Nature Materials, Vol. 6, June 2007, 413-417

A wireless system has drawbacks in that power needs to be remotely located in the wound and could limit the number and type of transducers that could be driven and communications protocols are more complex. Large, bariatric patients present a significant challenge from a communications perspective due to influence on signal strength and, therefore, a wire based system may have distinct advantages.

Compliance is another aspect that may be incorporated into the present invention. Maintaining negative pressure as specified by the caregiver over time is a significant aspect that contributes to the success of negative pressure therapy in healing difficult to treat wounds. Consistent application of negative pressure is termed “compliance” and has been treated in, for example, U.S. Pat. No. 7,438,705 (Karpowicz et al.) patent applications: System for Treating a Wound with Suction and Method of Detecting Loss of Suction (U.S. application Ser. No. 11/268,212), U.S. Patent Application Publication No. 2007/0218532, and U.S. Patent Application Publication No. 2008/0005000 (Radl et al.).

Compliance is further enhanced by the implementation of the invention described herein by eliminating the faults associated with remote instrumentation utilizing pressure signals conveyed via tubing. Pressure signals are subject to line losses and obstructions that may lead to false positive or false negative indications of the true status of the environmental condition of the wound.

Compliance is further enhanced in that the wound therapy controller 5 maintains an on board record of compliance over time. This data record can be downloaded to an external memory drive 10 (see FIG. 1). The data contained on drive 10 can be further transferred to a computer for formatting and record keeping. The data can be configured as a report that will indicate the complete compliance of the patient and device or a given time period. This information can be very useful in determining the potential cause of non-compliance.

Obtaining information on the status of the microenvironment enables a determination that the therapy is in fact effective and will lead to a predictable, beneficial outcome. Currently, the practice for medical therapy reimbursement involves paying for the therapy regardless of whether the therapy has been effective. Trends in insurance industries are increasingly seeking “pay for performance” types of remuneration. This type of payment at its most basic level involves a payment when and if a therapy is confirmed as being merely delivered. For example, effective therapy measured by the volume of oxygen consumed is not provided when the oxygen tank is turned on, but the breathing apparatus is not secured properly to the patient and oxygen is being pumped into the room. It is obvious that there is a distinct lack of “performance” in this case. Regarding the invention herein, the inventors have anticipated that the delivery of effective level of negative pressure to a wound should be monitored as delivering the level of negative pressure that is considered therapeutic and compliant and the system's ability to track compliant time is reflective of a “pay for performance” reimbursement model. The invention described herein, anticipates a more comprehensive level of compliance, one that not only recognizes that the device is turned on and is providing the therapy within prescribed ranges, but one where the status of the effectiveness of the therapy is ascertained and communicated to the clinician. Reimbursement may then be based on a prediction of a successful outcome. If a successful outcome is not predicted, then the therapy should be discontinued until the cause of the lack of progress is determined and corrected. Such causes may include nutritional defects, infection, or true lack of patient compliance in maintaining the therapy.

Complaint therapy is defined as a period of time wherein clinically effective levels of suction are delivered to the patient's wound bed. Instantaneous compliance is any period wherein the suction delivered is preferably within 10% of the medically prescribed set point pressure. Instantaneous compliance can also be a period wherein suction delivered is preferably within 25% of the medically prescribed set point. Noncompliant therapy time would be any time the suction at the wound is no within 25% of the clinically prescribed level.

Typical dressing changes utilizing negative pressure wound therapy occur every 48 to 72 hours. The present invention preferably uses a time based dimensionless number based in part on the overall patient compliance with therapy. This may be shown to the patient in a fractional format such as 21/24 hours, 42/48 hours, 63/72 hours or it may be displayed as a colored indicator to the patient. The indicator would preferable show acceptable compliance as the dimensionless number equals or exceeds 21/24 hours, 42/48 hours or 63/72 hours. Marginal compliance would be indicated and time compliance is greater than 15/25 hours, 30/48 hours, 45/72 hours, but less than the requisite number needed or acceptable compliance. Unacceptable compliance would be indicated any time the dimensionless number is less than marginal compliance. This dimensionless number provides a backward looking indicator of patient compliance that is readily understandable to patient and clinician alike. A report can be generated that when specific non-compliant events occur for the purpose of troubleshooting, training, etc.

In yet another aspect of the present invention, proper wound healing benefits from the maintenance of a moist wound healing environment. Cell migration and proliferation is dependent on surface moisture and films that are supportive of the natural healing process.

Wounds that lack surface moisture may be typified by eschar on the wound surface, and will not progress to full healing without proper intervention. Wounds that have too much surface moisture are typified by maceration of the wound bed and surrounding wound margins. These wounds may actually regress without proper clinical intervention. The goal of wound moisturization is to achieve an optimal healing balance between these wet and dry states.

Moisture at the wound bed can be read by a sensor inside of the dressing, or by the humidified air drawn into the system pump, as discussed above. Based on information from these readings, the system can make adjustments to the localized wound conditions to achieve an optimal balance. In one instance, an additional line directed to the wound site can instill an isotonic solution to increase humidity, or it can instill dehumidified room air in an attempt to reduce moisture on the wound bed.

In another aspect of the present invention, NPWT systems typically employ a hydrophobic membrane to prevent patient contaminated fluids from reaching the internal pump. These membranes will prevent liquids from entering the pump, but a number of important gaseous biological markers can readily pass through this membrane. Water vapor readily passes through a hydrophobic membrane and may be read with a humidity sensor to assess the level of hydration at the wound. Hydrogen sulfide will also be communicated past the membrane and its presence is indicative of tissue degradation and bacterial activity at the wound site. Methane, produced by anaerobic bacteria active at the wound bed can be monitored in the wound therapy controller to determine any increase in activity at the wound bed. Based on the information from the gas sampling analyzer in the wound therapy controller, the caregiver can be prompted to administer an appropriate regimen to change the level of moisture at the wound, or to appropriately administer antibiotic agents to handle aerobic or anaerobic bacterial colonization.

All references cited herein are incorporated herein by reference in their entireties.

While the invention has been described in detail and with reference to specific examples thereof, it will be apparent to one skilled in the art that various changes and modifications can be made therein without departing from the spirit and scope thereof.

What is claimed is:

1. A wound therapy system, comprising:
(a) at least one sensor, placed in a wound, for sensing information regarding status of a body of a living being; and
(b) a communication link for electronically passing the information regarding said at least one sensor to a controller.

2. The wound therapy system of claim 1, wherein the at least one sensor is at least one sensor selected from the group of a pressure sensor to sense pressure, a suction sensor for sensing suction, a temperature sensor to sense temperature, a relative humidity sensor to sense relative humidity, a pH sensor to sense a level of pH, an oxygen sensor to sense a level of oxygen and a CO₂ sensor to sense a level of CO₂, and a blood flow sensor for monitoring blood flow in tissue at the surface of the wound.

3. The wound therapy system of claim 1, wherein the controller provides real time monitoring of conditions sensed by the at least one sensor in the wound.

4. The wound therapy system of claim 3, wherein the controller controls a level of negative pressure applied to the wound.

5. The wound therapy system of claim 1, wherein one of the at least one sensor is a sensor for quantifying the presence of beneficial metabolites as an indicator of normal wound healing.

6. The wound therapy system of claim 5, including a pump and a conduit from the pump to the wound for adding metabolites.

7. The wound therapy system of claim 6, wherein said information is used in a feedback system in the controller to control the pump for adding metabolites based on information from the at least one sensor.

8. The wound therapy system of claim 6, including a solenoid used in setting a timed rate of infusion.

9. The wound therapy system of claim 1, wherein the at least one sensor is a collagen sensor for quantifying the rate of tissue regeneration in the wound.

10. The wound therapy system of claim 1, wherein one of the at least one sensor is a sensor for quantifying the presence of detrimental metabolites as an indicator of impaired wound healing or as an indicator of urgent action required.

11. The wound therapy system of claim 1, wherein one of the at least one sensor is an organic compound sensor.

12. The wound therapy system of claim 1, wherein one of the at least one sensor is an inorganic compound sensor.

13. The wound therapy system of claim 1, wherein the communication link is a wireless transmitter.

14. The wound therapy system of claim 1, wherein the communication link transmits the information via wires.

15. The wound therapy system of claim 1, where the at least one sensor is an array of sensors.

16. The wound therapy system of claim 1, wherein the controller is capable of receiving and interpreting signals from a global positioning system to allow it to determine an instantaneous location of the controller.

17. A wound therapy system, comprising:
(a) at least one sensor for sensing information regarding status of a body of a living being;
(b) a controller for monitoring and interpreting said information wherein said information is a pressure in the wound;
(c) a communication link for electronically passing the pressure in the wound to the controller, wherein the controller includes a transmitter to communicate the information to a central server.

18. The wound therapy system of claim 17, wherein said information is the pressure that has been maintained in the wound over a predetermined period of time.

19. The wound therapy system of claim 17, wherein the transmitter is a wireless transmitter.

20. The wound therapy system of claim 17, wherein the patient information transmitted to the central server is information selected from the group consisting of instantaneous patient compliance, historical patient compliance, error messages, and information related to service.

21. The wound therapy system of claim 1, wherein the controller includes a receiver to receive downloaded information from a central server.

22. The wound therapy system of claim 21, wherein the downloaded information is information selected from the group consisting of updated operating software, clinical operating protocols, and user defined settings.

23. The wound therapy system of claim 17, wherein the transmitter communicates with the central server to relay compliance time, wherein the compliance time is a period of time wherein clinically effective levels of suction are delivered to the patient's wound bed.

24. A method for providing wound therapy, comprising:
(a) providing at least one sensor, each sensor placed in a wound;
(b) sensing information regarding status of a body of a living being utilizing the at least one sensor; and
(c) passing the information from the at least one sensor to a controller via a communication link between the at least one sensor and the controller.

25. The method for providing wound therapy of claim 24, wherein the step of providing at least one sensor includes providing at least one sensor selected from the group of a pressure sensor to sense pressure, a suction sensor for sensing suction, a temperature sensor to sense temperature, a relative humidity sensor to sense relative humidity, a pH sensor to sense a level of pH, an oxygen sensor to sense a level of oxygen and a CO₂ sensor to sense a level of CO₂, and a blood flow sensor for monitoring blood flow in tissue at the surface of the wound.

26. The method for providing wound therapy of claim 24, including the step of real time monitoring of conditions sensed by the at least one sensor in the wound.

27. The method for providing wound therapy of claim 24, wherein the step of providing at least one sensor includes providing a sensor for quantifying the presence of beneficial metabolites as an indicator of normal wound healing.

28. The method for providing wound therapy of claim 27, including the step of adding metabolites by pumping the metabolites through a conduit to the wound.

29. The method for providing wound therapy of claim 28, including providing a feedback system in the controller wherein the information is used in a feedback system in the controller to pump the metabolites based on information from the at least one sensor.

30. The method for providing wound therapy of claim 28, including the step of setting a timed rate of infusion of by pumping.

31. The method for providing wound therapy of claim 24, wherein the step of providing least one sensor includes providing a collagen sensor for quantifying the rate of tissue regeneration in the wound.

32. The method for providing wound therapy of claim 24, wherein the step of providing one of the at least one sensor includes providing a sensor for quantifying the presence of detrimental metabolites as an indicator of impaired wound healing or as an indicator of urgent action required.

33. The method for providing wound therapy of claim 24, wherein the step of providing one of the at least one sensor includes providing an organic compound sensor.

34. The method for providing wound therapy of claim 24, wherein the step of providing one of the at least one sensor includes providing an inorganic compound sensor.

35. The method for providing wound therapy of claim 24, including the step of wirelessly transmitting the information from the at least one sensor to the controller.

36. The method for providing wound therapy of claim 24, including the step of passing the information from the at least one sensor to the controller via wires.

37. The method for providing wound therapy of claim 24, where the step of providing at least one sensor includes providing an array of sensors.

38. The method for providing wound therapy of claim 24, including the steps of providing the controller which is capable of receiving and interpreting signals from a global positioning system and determining an instantaneous location of the controller.

39. A method for providing wound therapy, comprising:
(a) providing at least one sensor;
(b) sensing information regarding status of a body of a living being utilizing the at least one sensor;
(c) passing the information from the at least one sensor to a controller via a communication link between the at least one sensor and the controller, wherein the controller is for monitoring and interpreting said information wherein said information is the pressure in the wound; and
(d) transmitting patient information from the controller to a central server.

40. The method of claim 39, wherein the information is a pressure that has been maintained in the wound over a predetermined period of time

41. The method for providing wound therapy of claim 39, wherein the step of transmitting includes transmitting at least one of instantaneous patient compliance, historical patient compliance, error messages, and controller or sensor service.

42. The method for providing wound therapy of claim 24, including the step of downloading information from a central server to the controller.

43. The method for providing wound therapy of claim 42, wherein the downloaded information is information selected from the group consisting of updated operating software, clinical operating protocols, and user defined settings.

44. The method for providing wound therapy of claim 39, including the step of transmitting compliance time, wherein the compliance time is a period of time wherein clinically effective levels of suction are delivered to the patient's wound bed.

45. The method for providing wound therapy of claim 44, wherein the step of transmitting compliance time includes transmitting a value equal to compliance time divided by a selected time period where the wound therapy system is operating.
